(19) United States
(12) Patent Application Publication
(10) Pub. No.: US 2015/0320887 A1
(43) Pub. Date: Nov. 12, 2015

(54) METHOD FOR DIAGNOSING GLUTEN SENSITIVITY AND USES THEREOF

(71) Applicant: FONDAZIONE IRCCS "CA' GRANDA - OSPEDALE MAGGIORE POLICLINICO", Milano (IT)

(72) Inventors: Luca Elli, Milano (IT); Carolina Tomba, Cassina de' Pecchi (IT); Leda Roncoroni, Milano (IT); Maria Teresa Bardella, Milano (IT)

(73) Assignee: FONDAZIONE IRCCS "CA' GRANDA-OSPEDALE MAGGIORE POLICLINICO", Milano (IT)

(21) Appl. No.: 14/443,081

(22) PCT Filed: Oct. 28, 2013
(86) PCT No.: PCT/IB2013/059721

§ 371 Date: May 15, 2015

(30) Foreign Application Priority Data
Nov. 15, 2012 (IT) MI2012A001932

(51) Int. Cl. A61K 49/00

(52) U.S. Cl. CPC A61K 49/0004

(57) ABSTRACT

The present invention relates to a method for determining gluten sensitivity which uses a kit comprising: a kit identification code, at least one oral formulation of gluten and at least one oral formulation of placebo, each identified by a code, for random double-blind administration. The method provides for at least one step of taking said at least one oral formulation of gluten and said at least one oral formulation of placebo according to a double-blind protocol; and at least one step of collecting data relating to the heath status of said subject.

### The present invention relates to a method for determining whether a subject is sensitive to gluten. Furthermore, the present invention regards a kit for carrying out said method.

Gluten is a very compact, elastic, porous lipoprotein substance present in wheat, barley, rye and several other graminaceous plants. The protein fraction of gluten contains gliadin and glutenin, which represent the toxic component of gluten. In fact, these proteins comprise the amino acid proline, in particular glutamine-proline repetitions, which are not easily digested by the human body.

Following the intake of gluten by a sensitive subject, as a result of intolerance, allergy or sensitivity to this substance, pathological conditions may arise which, despite manifesting roughly the same signs and the same symptoms, are clinically very different.

Celiac disease is a genetically based gluten intolerance associated with the markers DQ2 and DQ8 of the HLA system. This pathology manifests itself with immune-mediated damage to the intestinal mucosa and the appearance of specific serological markers, in particular anti-transglutaminase (tTG) antibodies, IgA class anti-endomysial antibodies (EMA) and IgA and IgG class anti-deamidated gliadin antibodies (AGA). The association between celiac disease and these specific antibodies is at the basis of the diagnostic technique of this pathology, which is in fact diagnosed by means of serological assays of the tTG, EMA and AGA antibodies.

Gluten allergy (also known wheat allergy) is a violent reaction of IgE to gliadin. This pathology manifests itself clinically with respiratory tract problems or anaphylactic reactions. At presence, the diagnosis of gluten allergy is based on prick tests and specific RAST tests.

Non-celiac gluten sensitivity, or more simply gluten sensitivity, is a recently identified pathological condition correlated with the intake of gluten. It manifests itself with both gastroenterological symptoms (bloating, diarrhoea and abdominal pain), and extra-enteric symptoms (headache, joint and muscle pains, blurred vision) which, in large part, are comparable to the symptoms of celiac disease or irritable bowel syndrome. However, unlike in the latter cases, a subject with gluten sensitivity does not show any atrophy of the intestinal villi, or an autoimmune response.

In London in 2011 there was the first Consensus Conference on gluten sensitivity, which, among other things, sought to give a definition to this condition. Unfortunately, the scant knowledge regarding the etiology of gluten sensitivity and the symptoms shared with other pathologies has also made it difficult to define this condition. In fact, there exists no definition of what gluten sensitivity is; rather, what it is not has been defined. In other words, at present, a subject is considered likely to be sensitive to gluten when he/she tests negative both for the specific serological markers of celiac disease, and the specific IgE of gluten allergy and, moreover, when an intestinal biopsy is within the normal range, i.e. without any villous atrophy.

Therefore, at present it is possible to determine whether a subject is sensitive to gluten only by process of elimination.

However, there is a greatly felt need to define a protocol for determining gluten sensitivity in a subject, since approximately 6%-14% of the population suffers from this pathology and therefore the problem is highly relevant from an epidemiological standpoint.

The present invention has resolved the above-described technical problem with a method for determining gluten sensitivity and a kit for carrying out said method as specified in the appended independent claims.

The preferred aspects of the present invention are specified in the appended dependent claims.

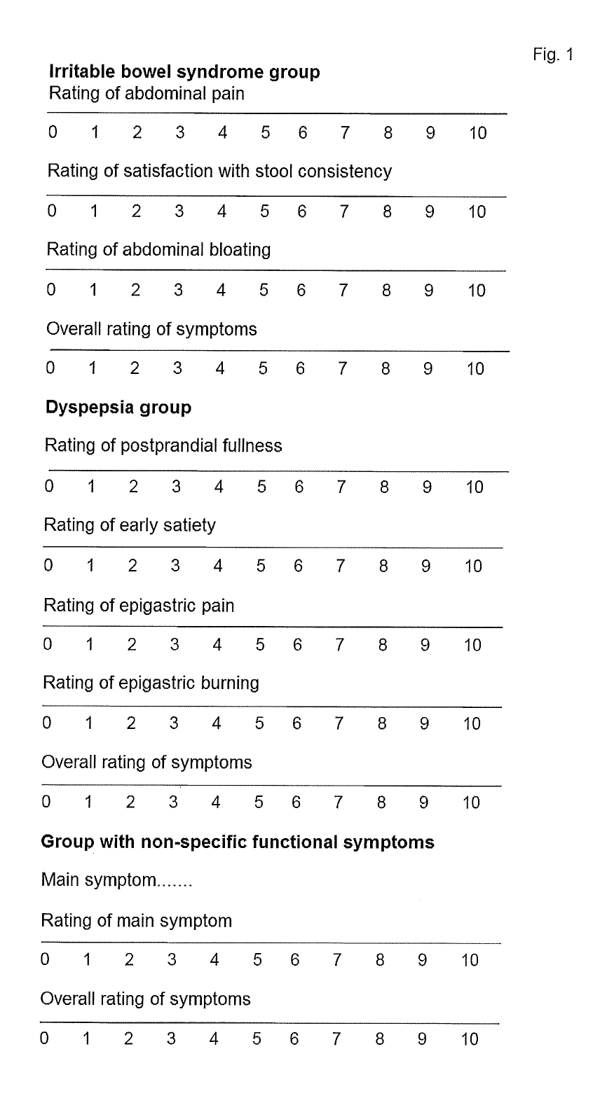

The present invention will be described below in detail, also with the aid of FIG. 1, which refers to an example of an ad hoc questionnaire for collecting data regarding the health status of a subject who carries out the method of the invention.

A first aspect of the present invention relates to a method for determining gluten sensitivity in a subject, said method comprising at least a step of:
providing said subject with a kit comprising: a kit identification code, at least one oral formulation of gluten in a quantity comprised between 4 and 10 grams, at least one oral formulation of placebo in a quantity comprised between 4 and 10 grams, said oral formulation of gluten and said oral formulation of placebo being intended for double-blind administration and each being identified by a code;
taking at least one said oral formulation of gluten and at least one said oral formulation of placebo in any order according to a double-blind protocol;
collecting data regarding the health status of said subject.

The kit that is provided to the subject for the purpose of carrying out the method of the present invention comprises (i) at least one oral formulation of gluten, i.e. a quantity of gluten comprised between 4 and 10 grams and formulated for oral administration, (ii) and at least one oral formulation of placebo, i.e. a quantity of placebo which ranges between 4 and 10 grams, wherein the gluten and the placebo are formulated for double-blind oral administration, i.e. the two oral formulations are identical in aesthetic appearance, that is, in form, but differ in substance, because one contains gluten and the other contains placebo. Moreover, each of the two formulations is identified by a code.

In this manner, the at least one oral formulation of gluten and the at least one oral formulation of placebo contained in a kit are such as to be indistinguishable by any subject. Moreover, the at least one oral formulation of gluten and the at least one oral formulation of placebo contained in the kit are univocally identified by any code whatsoever, for example a colour code, a numerical code, an alphabetic or alphanumeric code.

The correspondence of this code with the nature of the substance, i.e. whether it is an oral formulation containing gluten or an oral formulation containing placebo, is known only to the kit manufacturer. In fact, the kit is characterized by an identification code, based on which it is possible to determine the nature of the oral formulations that a subject has taken (i.e. gluten or placebo) because the manufacturer associated (at the time of production/sale) the identification code of the kit with each code of the oral formulations and hence the corresponding substances contained in each oral formulation (i.e. gluten or placebo). In this manner, it will be possible, after carrying out the method, to contact the manufacturer, for example by telephone or via web, specify the identification code of the kit, determine the nature of each oral formulation taken and establish the chronology of intake. In other words, it will be possible afterwards to distinguish the nature of the substance taken. In fact, the at least one oral formulation of gluten and the at least one oral formulation of placebo are identified by a code with the aim of enabling the correct association by the manufacturer. For example, the manufacturer can produce a kit with a certain identification code X in which the oral formulation of gluten has a code A, whereas the oral formulation of placebo in the same kit has a code B. The manufacturer records, in a database, that the kit having the identification code X has an oral formulation of gluten which corresponds to code A and an oral formulation of placebo which corresponds to code B. In all other respects the oral formulations are identical, except in content, which is obviously not visible. At the end of the period of intake, the subject will contact the manufacturer in order to associate, via the identification code of the kit, the code of each oral formulation with the substance contained therein and consequently establish the chronology of the intake, that is, if he/she took the gluten first and then the placebo or vice versa.

In the context of the present invention, the term “double blind” means that neither the subject, nor whoever suggests carrying out the method (generally a physician) is aware of when gluten or the placebo is taken. Said at least one oral formulation of gluten and said at least one oral formulation of placebo are preferably in the form of pastilles, pills, capsules or tablets.

Preferably, each kit comprises at least one, preferably at least five, more preferably at least seven pastilles, pills, capsules or tablets of gluten and at least one, preferably at least five, more preferably at least seven pastilles, pills, capsules or tablets of placebo. Preferably, the number of the oral formulations of gluten and the number of oral formulations of placebo for each kit is the same.

In fact, the step of taking at least one oral formulation of gluten and the step of taking at least one oral formulation of placebo on a double-blind basis comprises at least one intake, preferably at least seven intakes. Preferably, said at least one step of taking at least one oral formulation of gluten is carried out before or after said at least one step of taking at least one oral formulation of placebo.

Therefore, in particular, the method of determining gluten sensitivity according to the present invention envisages that the subject takes gluten for a week and takes the placebo for a week or vice-versa; the intake of gluten and/or placebo takes place randomly, on a double-blind basis.

Preferably, a wash-out phase takes place between said at least one step of taking at least one oral formulation of gluten and said at least one step of taking at least one oral formulation of placebo. Said wash-out phase preferably lasts for at least five days, preferably for at least seven days, more preferably for seven days.

In the context of the present invention, the term wash-out means a period between two periods of taking gluten or placebo in which the subject takes nothing and should thus “expel” everything he/she took in previously.

The particularly preferred embodiment of the method according to the present invention envisages: at least one step of a subject taking at least one oral formulation of gluten or at least one of said oral formulation of placebo for a week; and/or at least a wash-out phase of one week in which said subject takes neither the oral gluten formulation nor the oral formulation of placebo; and/or at least one step in which said subject takes at least one oral formulation of placebo or at least one of said oral formulation of gluten for a week.

Alternatively, the subject can take the placebo for a week, then undergo a week of wash-out and finally take gluten for another week. The intake step is carried out randomly on a double-blind basis, so the subject does not know which substance he/she is taking.

Said placebo is preferably selected from among: rice starch and other inert molecules, possibly also in different dosages by weight.

According to a preferred embodiment, the subject to whom the method of the invention is applied previously followed a gluten-free diet.

The step of collecting data regarding the health status of the subject is preferably carried out by collecting said data in a questionnaire. Said questionnaire is prepared ad hoc to collect data regarding the health status of a subject who carries out the method of the invention. In particular, said questionnaire is a standard form which includes questions related to the health status of said subject. The subject can respond using the evaluation scales associated with each question. The evaluation scale is preferably a verbal numerical scale (VNS), or a visual analogue scale (VAS) or a verbal rating scale (VRS).

Preferably, data is collected by the subject via Web at the beginning and at the end of said at least one step of taking at least one oral formulation of gluten and/or at the start and at the end of at least one step of taking at least one oral formulation of placebo.

In particular, the questionnaire can contain questions about the health status of the subject which are related to the group to which the subject belongs. In particular, subjects can be divided into groups based on their symptoms. For example, the subjects who carry out the method according to the present invention can be divided into a group with irritable bowel syndrome or a group with symptoms of a dyspeptic, diarrhoeic type, with alterations of the bowel or extraintestinal manifestations such as mild neurological or dermatological manifestations, or into a group that exhibits non-specific symptoms.

The questions can be specific for each group.

An example of a questionnaire is shown in FIG. 1, where different questions are proposed for the various groups of subjects with a numerical rating scale in which the subject will indicate a value corresponding to the specific health status required in the question.

Applying the proposed method, it has been observed that a worsening in at least 30% of the data regarding a subject's health status, in particular the main symptoms, occurring during said at least one step of administering at least one oral formulation of gluten, makes it possible to say that the subject is sensitive to gluten.

Therefore, a further aspect of the present invention relates to the use of said kit for diagnostic purposes. In particular, the kit is used to determine gluten sensitivity in a subject. Preferably, the determination of gluten sensitivity in said subject is achieved by applying the method of the present invention.

A further aspect of the present invention regards a kit for carrying out the method to determine gluten sensitivity according to the present invention, said kit comprising:
a kit identification code;
at least one oral formulation of gluten in a quantity comprised between 4 and 10 grams;
at least one oral formulation of placebo in a quantity comprised between 4 and 10 grams;
   said oral formulation of gluten and said oral formulation of placebo being for double-blind administration and each being identified by a code.

Preferably, said at least one oral formulation of gluten and said at least one oral formulation of placebo is at least five, preferably seven formulations. In a particularly preferred embodiment, the number of oral formulations of said gluten is equal to the number of oral formulations of said placebo. For example, the kit comprising at least seven oral formulations of gluten and at least seven oral formulations of placebo is useful, for example, for enabling a weekly intake of said oral formulations.

According to a preferred embodiment of the invention, the at least one oral formulation consists of at least one pill, at least one pastille, at least one lozenge, or at least one tablet.

Said oral formulations are identified by a code, for example a colour code, a numerical code, an alphabetic code, or an alphanumeric code. For the purpose of the method, said code will serve to understand when gluten was taken and when the placebo was taken, as earlier described.

According to a further embodiment of the present invention, the kit further comprises an ad hoc questionnaire prepared to collect data regarding the health status of a subject who carries out the method of the invention.

In particular, said questionnaire is a standard form containing questions related to the health status of said subject. The subject can answer using the evaluation scale associated with each question. The evaluation scale is preferably a verbal numerical scale (VNS), a visual analogue scale (VAS) or a verbal rating scale (VRS).

1. A kit for determining gluten sensitivity in a subject, said kit comprising:
a kit identification code;
at least one oral formulation of gluten in a quantity comprised between 4 and 10 grams;
at least one oral formulation of placebo in a quantity comprised between 4 and 10 grams;
said at least one oral formulation of gluten and at least one oral formulation of placebo being for double-blind administration and each being identified by a code.

2. The kit according to claim 1, comprising an equal number of said at least one oral formulation of gluten and of said at least one oral formulation of placebo.

3. The kit according to claim 1, wherein said at least one oral formulation is at least one pill, at least one pastille, at least one lozenge, or at least one tablet.

4. The kit according to claim 1, wherein said placebo is rice starch.

5. The kit according to claim 1, further comprising a questionnaire prepared ad hoc for the collection, of data referring to the health status of said subject.

6. The kit according to claim 5, wherein said questionnaire comprises at least one rating scale.

7. A method for determining gluten sensitivity in a subject, said method comprising at least one step of:
providing said subject with a kit according to claim 1;
taking at least one of said oral formulation of gluten and at least one of said oral formulation of placebo in any order according to a double-blind protocol;
collecting the data referring to the health status of said subject.

8. The method according to claim 7, wherein said step of taking at least one of said oral formulation of gluten is performed before or after said step of taking at least one of said oral formulation of placebo.

9. The method according to claim 7, wherein at least one wash-out phase takes place between said step of taking at least one of said oral formulation of gluten and said step of taking at least one oral formulation of placebo.

10. The method according to claim 9, wherein said step of taking at least one oral formulation of gluten, and/or said step of taking at least one oral formulation of placebo, and/or said at least one wash-out phase takes place over a period of at least five days.

11. The method according to claim 7, comprising at least one step wherein said subject takes at least one oral formulation of gluten or at least one oral formulation of placebo for a week; and/or at least one week-long wash-out phase wherein said subject takes neither the oral formulation of gluten, nor the oral formulation of placebo; and/or at least one step wherein said subject takes at least one oral formulation of placebo or at least one oral formulation of gluten for a week.

12. The method according to claim 7, wherein said subject has previously followed a gluten-free diet.

13. The method according to claim 7, wherein said step of collecting data referring to the health status of the subject is performed by collecting said data in a questionnaire.

14. The method according to claim 13, wherein said questionnaire is a standard form containing questions about the health status of a subject, wherein said questions are associated with a rating scale.

15. The method according to claim 7, wherein said data collection step is performed by the subject at the start and at the end of said at least one step of taking at least one oral formulation of gluten and/or at the start and at the end of said at least one step of taking at least one oral formulation of placebo.

16. (canceled)

17. The kit according to claim 6, wherein said rating scale is selected from the group consisting essentially of a verbal numerical scale, a visual analogue scale and a verbal rating scale.

18. The method according to claim 14, wherein said rating scale is selected from the group consisting essentially of a verbal numerical scale, a visual analogue scale and a verbal rating scale.
